(19) United States
(12) Patent Application Publication
(10) Pub. No.: US 2026/0127954 A1
(43) Pub. Date: May 7, 2026

(54) INFORMATION PROCESSING SYSTEM, INFORMATION PROCESSING METHOD, AND NON-TRANSITORY COMPUTER READABLE MEDIUM

(71) Applicant: FUJIFILM Business Innovation Corp., Tokyo (JP)

(72) Inventor: Shohei KAWAKAMI, Kanagawa (JP)

(73) Assignee: FUJIFILM Business Innovation Corp., Tokyo (JP)

(21) Appl. No.: 19/204,791

(22) Filed: May 12, 2025

(30) Foreign Application Priority Data
Nov. 5, 2024 (JP) 2024-193398

(51) Int. Cl. G08B 21/04; A61B 5/11; G06T 7/70

(52) U.S. Cl. CPC G08B 21/043; A61B 5/1117; G06T 7/70

(57) ABSTRACT

An information processing system includes a processor configured to: continuously capture images that include a monitored person; perform skeletal estimation of the person in a captured image to detect whether or not the person is in an abnormal pose; if an abnormal pose is detected, input the image in which the abnormal pose of the person is detected into a large language model that converts the content of an inputted image into text information and outputs the text information, and thereby acquire text information pertaining to the state of the person in the inputted image; and provide a notification indicating that an abnormal pose of the monitored person has occurred, together with the acquired text information and the image in which the abnormal pose is detected, to a preset destination.

### CROSS-REFERENCE TO RELATED APPLICATIONS

This application is based on and claims priority under 35 USC 119 from Japanese Patent Application No. 2024-193398 filed Nov. 5, 2024.

### BACKGROUND

### (i) Technical Field

The present disclosure relates to an information processing system, an information processing method, and a non-transitory computer readable medium.

### (ii) Related Art

International Publication No. WO 2022/249635 discloses an action detection system that provides a notification regarding the detection of an action by a person included in an image to a prescribed destination when both of the following conditions are met: a first notification condition set specifically for an action class identified on the basis of the action; and a second notification condition, such as the duration of the action, the level of congestion around the person, and the time of day when the image was captured.

### SUMMARY

In recent years, the advancement of artificial intelligence (AI) technology has led to AI technology being utilized in various fields. For example, a proposed system uses a camera to capture a monitored person to be monitored and applies AI technology to the captured image to detect falls by the person. A goal in the fields of medicine and nursing care is to quickly notice a fall and issue an alert, leading to rapid aid efforts. In other cases, AI technology is being utilized to stop machines when a fall or other dangerous behavior is detected in places such as factories where dangerous work is performed.

However, even if an image of a monitored person is captured and a notification is provided to a preset destination upon detecting that the person in the captured image has fallen, a person who receives the notification may need to check the captured image, and may not be able to easily ascertain what kind of state the monitored person is in.

Aspects of non-limiting embodiments of the present disclosure relate to facilitating the ascertaining of a detected abnormal pose state, as compared to the case in which a monitored person is detected to be in an abnormal pose from a captured image of the person and only a notification is provided.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According to an aspect of the present disclosure, there is provided an information processing system including a processor configured to: continuously capture images that include a monitored person; perform skeletal estimation of the person in a captured image to detect whether or not the person is in an abnormal pose; if an abnormal pose is detected, input the image in which the abnormal pose of the person is detected into a large language model that converts the content of an inputted image into text information and outputs the text information, and thereby acquire text information pertaining to the state of the person in the inputted image; and provide a notification indicating that an abnormal pose of the monitored person has occurred, together with the acquired text information and the image in which the abnormal pose is detected, to a preset destination.

### BRIEF DESCRIPTION OF THE DRAWINGS

Exemplary embodiments of the present disclosure will be described in detail based on the following figures, wherein:

FIG. 1 is a diagram illustrating a system configuration of an information processing system according to an exemplary embodiment of the present disclosure;

FIG. 2 is a block diagram illustrating a hardware configuration of a management server 10 according to an exemplary embodiment of the present disclosure;

FIG. 3 is a block diagram illustrating a functional configuration of a management server 10 according to an exemplary embodiment of the present disclosure;

FIG. 4 is a diagram for explaining an example of processing in an LLM 16;

FIG. 5 is a flowchart for explaining operations by an information processing system according to an exemplary embodiment of the present disclosure;

FIG. 6 is a diagram illustrating an example of a notification in the case of an ongoing fallen state of a monitored person 21;

FIG. 7 is a diagram illustrating an example of a notification in the case where a monitored person 21 falls and then gets back up;

FIG. 8 is a flowchart for explaining operations by an information processing system in the case where an abnormal pose other than a fall is also detected and a notification is provided to a terminal device 40;

FIG. 9 is a diagram illustrating notification example 1 for the case where an abnormal pose other than a fall is detected; and

FIG. 10 is a diagram illustrating notification example 2 for the case where an abnormal pose other than a fall is detected.

### DETAILED DESCRIPTION

The following describes exemplary embodiments of the present disclosure in detail with reference to the drawings.

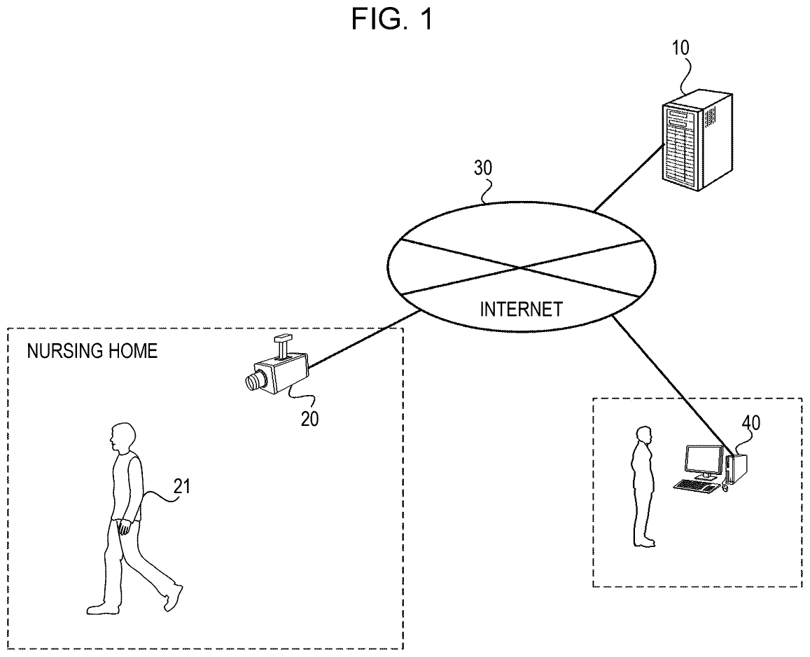

FIG. 1 is a diagram illustrating a system configuration of an information processing system according to an exemplary embodiment of the present disclosure.

An information processing system according to the exemplary embodiment enables a remote user to monitor the occurrence of an abnormal pose, such as a fall, of a monitored person 21 residing in a nursing home, for example. As illustrated in FIG. 1, the information processing system according to the exemplary embodiment includes a management server 10, a camera 20, and a terminal device 40 which are interconnected over a network such as the Internet 30. The camera 20 is installed in a nursing home and continuously captures images of actions by the monitored person 21. Images captured by the camera 20 are transmitted to the management server 10 over the Internet 30. In the management server 10, object detection processing involving AI technology is executed on images transmitted from the camera 20. If a person is detected to be present in an image as a result of the object detection processing, the management server 10 performs skeletal estimation processing on the area in which the person is detected to be present, acquires skeletal coordinate information, and detects whether or not the person is in an abnormal pose. If an abnormal pose, such as a fall, of the monitored person 21 is detected, the management server 10 provides a notification indicating that an abnormal pose of the monitored person 21 has occurred to the terminal device 40, which is used by a monitoring person.

Note that although the exemplary embodiment describes a configuration in which the processing for detecting an object in an image and the processing for skeletal estimation of a detected person are executed in the management server 10, the present disclosure is not limited to such a configuration. A configuration may also be adopted such that the functions of the management server 10 are provided in an on-premises server or an edge server. It is also possible to configure the camera 20 as an endpoint camera provided with the functions of the management server 10.

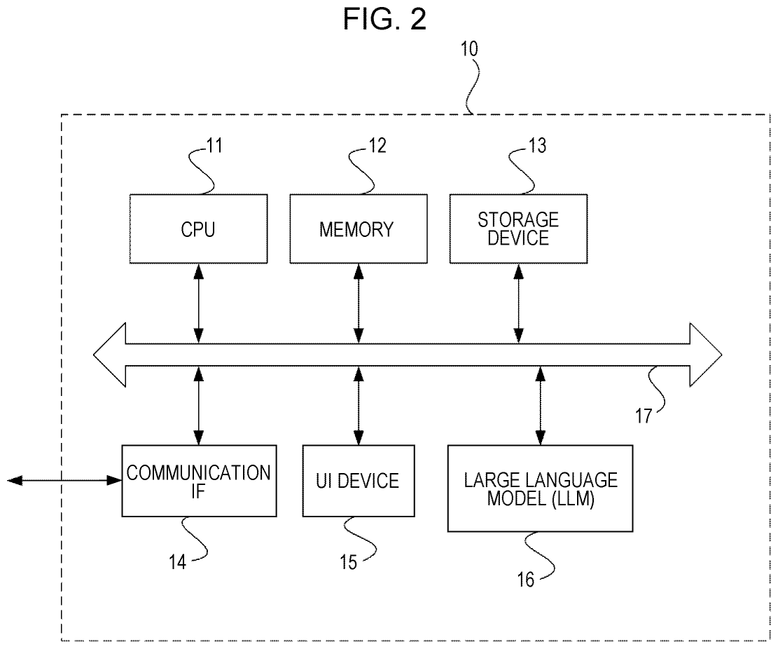

Next, a hardware configuration of the management server 10 in the information processing system according to the exemplary embodiment is illustrated in FIG. 2. As illustrated in FIG. 2, the management server 10 includes a CPU 11, a memory 12, a storage device 13 such as a hard disk drive, a communication interface (abbreviated as IF) 14 that transmits and receives data to and from an external device over the Internet 30, a user interface (abbreviated as UI) device 15 including a touch panel or a liquid crystal display and a keyboard, and a large language model (hereinafter abbreviated as LLM) 16. These components are interconnected through a control bus 17. Details of the LLM 16 will be described later.

The CPU 11 is a processor that controls operations by the management server 10 by executing predetermined processing on the basis of a control program stored in the memory 12 or the storage device 13. Note that although the CPU 11 is described as reading out and executing a control program stored in the memory 12 or the storage device 13 in the exemplary embodiment, the control program is not limited thereto. The control program may also be provided by being recorded onto a computer readable recording medium. For example, the program may be provided by being recorded on an optical disc, such as a Compact Disc-Read-Only Memory (CD-ROM) or a Digital Versatile Disc-Read-Only Memory (DVD-ROM), or by being recorded on a semiconductor memory, such as Universal Serial Bus (USB) memory or a memory card. The control program may also be acquired from an external device over a communication channel connected to the communication interface 14. Furthermore, for example, the control program may be provided as standalone application software, or the program may be incorporated into the software of each device as a function of the management server 10.

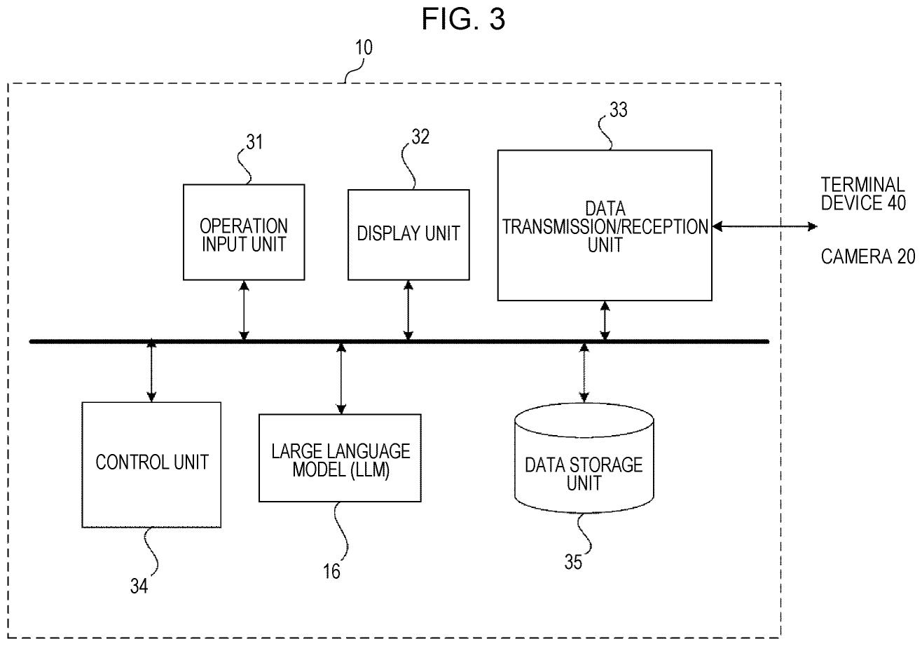

FIG. 3 is a block diagram illustrating a functional configuration of the management server 10 achieved by the execution of the above control program.

As illustrated in FIG. 3, the management server 10 according to the exemplary embodiment is provided with an operation input unit 31, a display unit 32, a data transmission/reception unit 33, a control unit 34, the LLM 16, and a data storage unit 35.

The data transmission/reception unit 33 transmits and receives data to and from external devices such as the terminal device 40 and the camera 20. The display unit 32 is controlled by the control unit 34 to display various information to the user. The operation input unit 31 inputs information on various operations performed by a user.

The control unit 34 receives captured image data from the camera 20 via the data transmission/reception unit 33, and upon detecting an abnormal pose of the monitored person 21 by using the received image data, carries out control to provide a notification to that effect to the terminal device 40. The data storage unit 35 stores various data, such as image data received by the data transmission/reception unit 33.

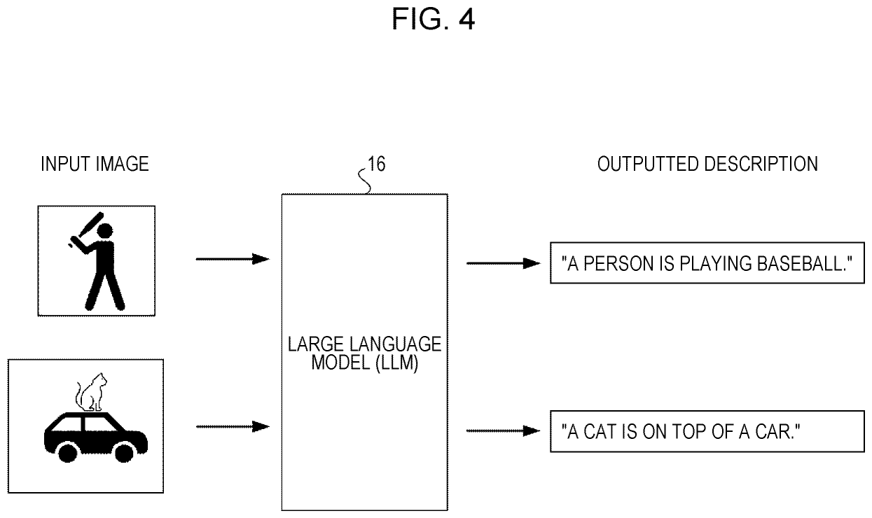

Next, an example of processing in the LLM 16 will be described with reference to FIG. 4.

The LLM 16 has a function of converting the content of an inputted image into text information and outputting the text information. For example, as illustrated in FIG. 4, if an image showing a person playing baseball is inputted into the LLM 16, the text information “A person is playing baseball.” is outputted. As another example, if an image of a cat sitting on top of a car is inputted into the LLM 16, the text information “A cat is on top of a car.” is outputted. In this way, an image may be inputted into the LLM 16 to obtain text information describing the content of the image.

In the information processing system according to the exemplary embodiment, by capturing an image of the monitored person 21 and applying AI technology to the captured image, an abnormal pose, such as a fall, of the person is detected, and a notification indicating that an abnormal pose of the monitored person 21 has occurred is provided to the terminal device 40.

However, even if a notification indicating that an abnormal pose of the monitored person 21 has occurred is provided to a preset destination such as the terminal device 40, the person who receives the notification may need to actually look at and check the captured image, and may not be able to easily ascertain what kind of state the monitored person 21 is in.

Accordingly, in the information processing system according to the exemplary embodiment, instead of simply providing a notification indicating that an abnormal pose of the monitored person 21 has occurred, the notification is provided together with text information acquired from the LLM 16 to facilitate the ascertaining of the detected abnormal pose state.

In the exemplary embodiment, the camera 20 continuously captures images that include the monitored person 21 and transmits the captured images to the management server 10.

In the management server 10, the control unit 34 performs skeletal estimation of the person in the captured image and detects whether or not the person is in an abnormal pose, such as a fall. Upon detecting an abnormal post of the monitored person 21, the control unit 34 inputs the image in which the monitored person 21 is detected to be in an abnormal pose into the LLM 16, and thereby acquires text information pertaining to the state of the person 21 in the inputted image. The control unit 34 then provides a notification indicating that an abnormal pose of the monitored person 21 has occurred, together with the text information acquired from the LLM 16 and the image in which the abnormal pose is detected, to a preset destination, namely the terminal device 40.

Note that the exemplary embodiment describes the case where the abnormal pose to be detected by skeletal estimation is a fall by the monitored person 21. However, the abnormal pose to be detected by skeletal estimation is not limited to a fall, and the abnormal pose also encompasses states other than a fall which are different from a normal state, such as a state of being slumped over a table and not moving or a state of leaning against a wall and not moving, for example.

Furthermore, upon detecting a fall by the monitored person 21, the control unit 34 inputs the image in which the fall by the monitored person 21 is detected and images before and after the fall into the LLM 16, and thereby acquires text information containing information pertaining to the state of the monitored person 21 before and after the fall. The control unit 34 may then provide to the terminal device 40 the acquired text information containing information pertaining to the state of the monitored person 21 before and after the fall, together with a notification indicating that an abnormal pose of the monitored person 21 has occurred, the image in which the abnormal pose is detected, and images before and after the fall.

A notification containing not only information about the time of the fall by the monitored person 21 but also information about before and after the fall is provided in this way because if the monitored person 21 gets up immediately after the fall, the urgency is not high, but if the monitored person 21 remains in a fallen state, it is highly likely that the urgency is high and a rapid response may be necessary.

Next, operations by the information processing system according to the exemplary embodiment will be described in detail with reference to the drawings.

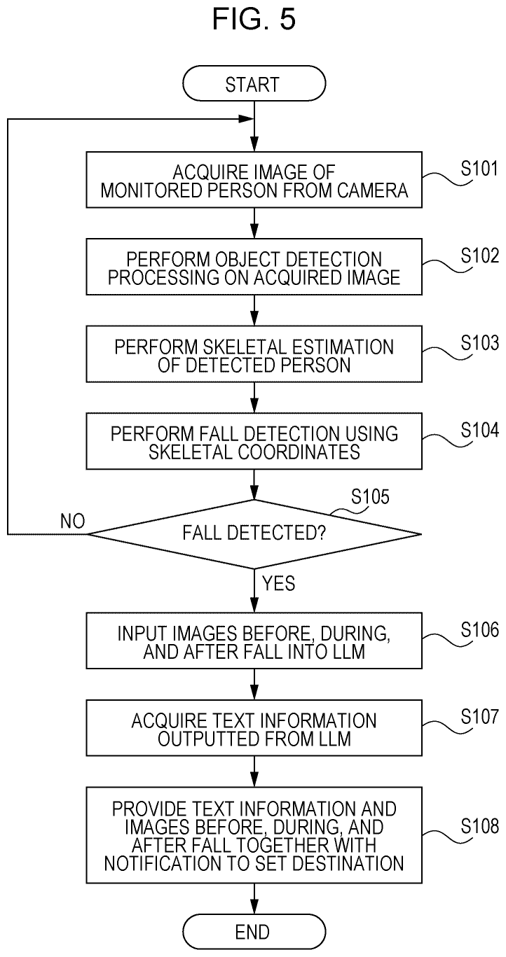

FIG. 5 is a flowchart for explaining operations by an information processing system according to the exemplary embodiment. The following describes a case of detecting a fall by the monitored person 21 and providing a notification indicating the detection to the terminal device 40.

First, in step S101, an image of the monitored person 21 is acquired by the camera 20. In step S102, the image acquired by the camera 20 is transmitted to the management server 10, and in the management server 10, object detection processing is performed on the transmitted image by the control unit 34.

In step S103, the control unit 34 performs skeletal estimation processing on a person detected in the image and acquires skeletal coordinate information. In step S104, the control unit 34 uses the acquired skeletal coordinate information to detect a fall by the person in the image.

Next, in step S105, it is determined whether or not a fall by the person in the image is detected. If a fall by the person in the image is not detected (step S105, no), the flow returns to the processing in step S101 and the processing in steps S101 to S104 is repeated. If a fall by the person in the image is detected (step S105, yes), in step S106, the control unit 34 inputs images before, during, and after the fall into the LLM 16. In step S107, the control unit 34 acquires text information outputted from the LLM 16.

Finally, in step S108, the control unit 34 provides the text information acquired from the LLM 16 and the images before, during, and after the fall, together with a notification indicating that the monitored person 21 fell down, to a set destination, namely the terminal device 40.

Note that if images captured by the camera 20 were inputted into the LLM 16 and converted to text information continuously, the amount of processing would be enormous. Accordingly, in the exemplary embodiment, an image in which a fall is detected is inputted into the LLM 16 and converted to text information only when a fall by the monitored person 21 is detected as described above, thereby reducing the amount of processing as compared to the case where captured images are inputted into the LLM 16 and converted to text continuously.

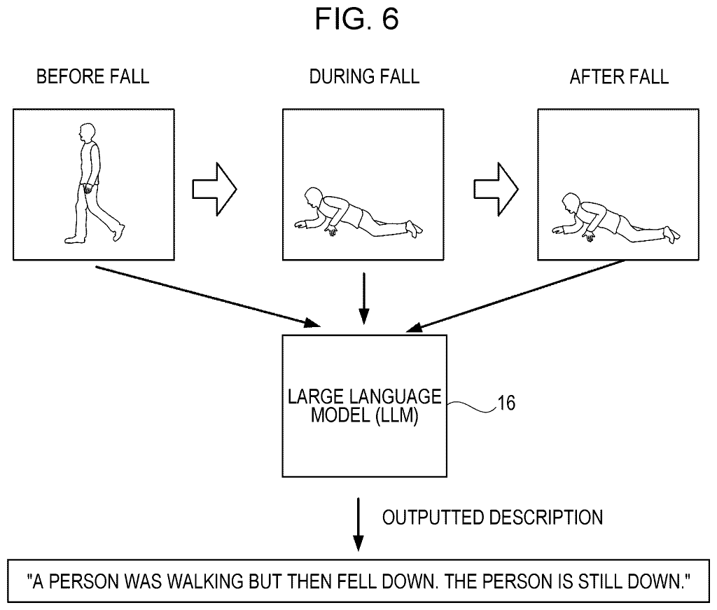

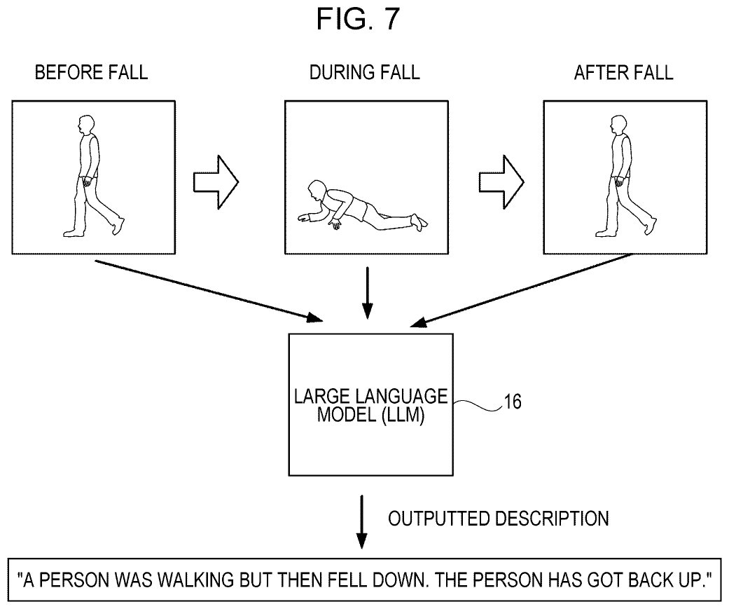

Next, FIGS. 6 and 7 illustrate examples of notifications provided to the terminal device 40 as a result of processing like the above. FIG. 6 illustrates an example of a notification in the case of an ongoing fallen state of the monitored person 21. FIG. 7 illustrates an example of a notification in the case where the monitored person 21 falls and then gets back up.

Referring to FIG. 6, the diagram illustrates a situation in which the person 21 who was previously walking normally falls, and remains in a fallen state without getting up after the fall. In such a case, inputting images before, during, and after the fall into the LLM 16 causes the LLM 16 to output the text information “A person was walking but then fell down. The person is still down.” Accordingly, the control unit 34 provides the images before, during, and after the fall and the text information “A person was walking but then fell down. The person is still down.” to the terminal device 40, together with a notification indicating that the monitored person 21 fell down. The user receiving such a notification is easily able to determine that the monitored person 21 has not got up after falling, and the urgency is high.

Referring to FIG. 7, the diagram illustrates a situation in which the person 21 who was previously walking normally falls, and gets up after the fall. In such a case, inputting images before, during, and after the fall into the LLM 16 causes the LLM 16 to output the text information “A person was walking but then fell down. The person has got back up.” Accordingly, the control unit 34 provides the images before, during, and after the fall and the text information “A person was walking but then fell down. The person has got back up.” to the terminal device 40, together with a notification indicating that the monitored person 21 fell down. The user receiving such a notification is easily able to determine that the monitored person 21 fell down but got up afterward, and the urgency is low.

### Exemplary Modification

Note that the above description uses the case of detecting that the monitored person 21 fell down and providing a notification to a preset destination, but a notification may also be provided to a preset destination in regard to an abnormal pose other than a fall.

For example, if an abnormal pose of the monitored person 21 is detected by skeletal estimation, the control unit 34 determines whether or not the detected abnormal pose is a fall by the monitored person 21. If the detected abnormal pose is determined not to be a fall by the monitored person 21, the control unit 34 inputs the image in which the abnormal pose is detected into the LLM 16, and thereby acquires text information pertaining to the state of the person in the inputted image. The control unit 34 then provides a notification indicating that an abnormal pose of the monitored person 21 has occurred, together with the acquired text information and the image in which the abnormal pose is detected, to a preset destination.

In such an exemplary modification, if the detected abnormal pose is a fall by the monitored person 21, the control unit 34 inputs the image in which the fall by the person 21 is detected and images before and after the fall into the LLM 16, and thereby acquires text information containing information pertaining to the state of the monitored person 21 before and after the fall. The control unit 34 then provides a notification indicating that a fall by the monitored person 21 has occurred, together with the acquired text information and the image in which the fall is detected, to a preset destination.

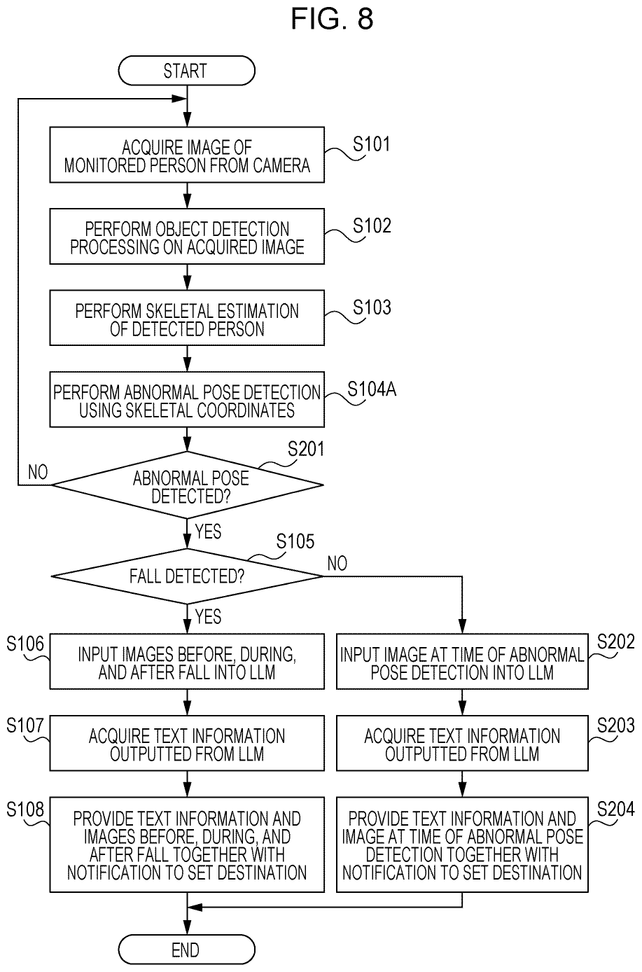

Next, the flowchart in FIG. 8 illustrates operations by the information processing system in the case where an abnormal pose other than a fall is also detected and a notification is provided to the terminal device 40. Note that in the flowchart in FIG. 8, processing which is the same as the flowchart in FIG. 5 is denoted with the same signs, and description thereof is reduced or omitted.

The flowchart in FIG. 8 is the flowchart illustrated in FIG. 5 with step S104A substituted for step S104 and with the addition of the processing in steps S201 to S204.

In such an exemplary modification, in step S104A, the control unit 34 uses acquired skeletal coordinate information to detect an abnormal pose, including a fall, of a person in an image.

In step S201, it is determined whether or not some kind of abnormal pose of the person in the image is detected. If an abnormal pose of the person in the image is not detected (step S201, no), the flow returns to the processing in step S101 and the processing in steps S101 to S104A is repeated. If some kind of abnormal pose of the person in the image is detected (step S201, yes), in step S105, the control unit 34 determines whether or not a fall by the person in the image is detected.

If it is determined that a fall by the person in the image is detected (step S105, yes), the control unit 34 executes the processing in steps S106 to S108, in a similar manner to that described above. If a fall by the person in the image is not detected (step S105, no), in step S202, the control unit 34 inputs the image at the time of the abnormal pose detection into the LLM 16. In step S203, the control unit 34 acquires text information outputted from the LLM 16. Finally, in step S204, the control unit 34 provides the text information acquired from the LLM 16 and the image at the time of the abnormal pose detection, together with a notification indicating that an abnormal pose of the monitored person 21 has occurred, to a set destination, namely the terminal device 40.

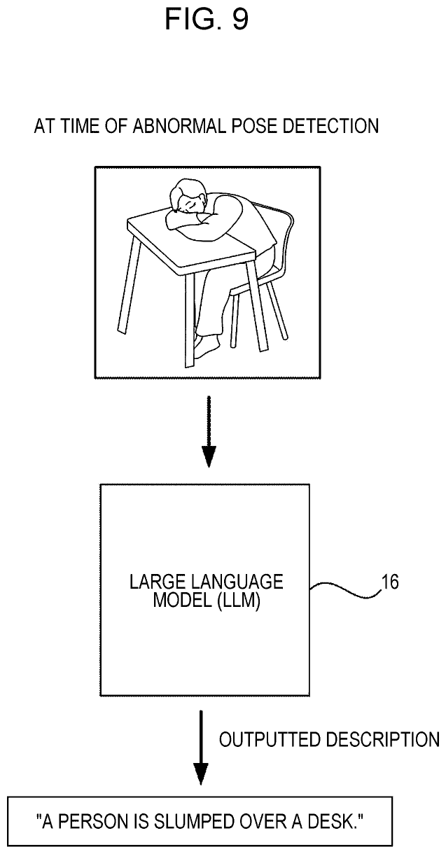

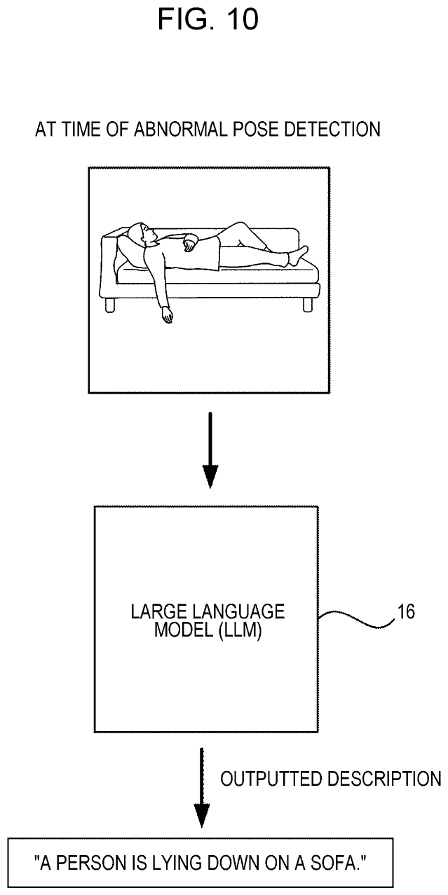

FIGS. 9 and 10 illustrate examples of notifications in the case where an abnormal pose other than a fall is detected by the processing described above.

FIG. 9 illustrates an example of a notification in the case where a state of the monitored person 21 slumped over a desk is detected as the abnormal pose. Referring to FIG. 9, inputting an image in which the abnormal pose is detected into the LLM 16 causes the LLM 16 to output, for example, the text information “A person is slumped over a desk.” Accordingly, the control unit 34 provides the image at the time of the abnormal pose detection and the text information “A person is slumped over a desk.” to the terminal device 40, together with a notification indicating that an abnormal pose of the monitored person 21 has occurred. The user receiving such a notification is easily able to determine what kind of abnormal pose of the monitored person 21 has occurred by simply reading the text information.

FIG. 10 illustrates an example of a notification in the case where a state of the monitored person 21 asleep on a sofa is detected as the abnormal pose. Referring to FIG. 10, inputting an image in which the abnormal pose is detected into the LLM 16 causes the LLM 16 to output, for example, the text information “A person is lying down on a sofa.” Accordingly, the control unit 34 provides the image at the time of the abnormal pose detection and the text information “A person is lying down on a sofa.” to the terminal device 40, together with a notification indicating that an abnormal pose of the monitored person 21 has occurred. The user receiving such a notification is easily able to determine what kind of abnormal pose of the monitored person 21 has occurred by simply reading the text information.

In the exemplary embodiment, processes are executed by a computer of any kind. The computer of any kind may execute these processes using a processor as hardware, by a program as software, or by a combination of the above. In the latter case, the processor is configured to cooperate with the program to execute various processes according to the exemplary embodiment, and may function as each unit or means according to the exemplary embodiment. The order in which operations are to be executed by the processor is not limited to the order described and may be changed, as appropriate. The computer of any kind may be a general-purpose computer, an application-specific computer, a workstation, or some other system capable of executing the processes.

The processor may be configured using one or multiple pieces of hardware, and the type of hardware is not limited. For example, the processor may be configured using hardware such as a central processing unit (CPU), a microprocessing unit (MPU), a programmable logic device such as a field-programmable gate array (FPGA), a special-purpose circuit such as an application-specific integrated circuit (ASIC) for executing specific processing, a graphics processing unit (GPU), or a neural processing unit (NPU). The type of hardware may also be a combination of different types of hardware. In the case where multiple pieces of hardware are configured to execute one or more processes of a certain processor, the multiple pieces of hardware may be present in physically discrete devices or may be present in the same device. Also, in any exemplary embodiments, the order of the processes by the processor is not limited to the order described above and may be changed, as appropriate. Note that hardware is formed from electrical circuitry or the like in which circuit elements such as semiconductor elements are combined.

Furthermore, the program may be software such as firmware or microcode. The program may also be a group of program modules, for example, each function of which may be achieved by a processor configured to execute the respective function. The program may also be program code and/or multiple code segments saved in one or more non-transitory computer readable media (for example, memory media and/or other forms of storage). The program may also be split and saved in multiple non-transitory computer readable media residing in physically discrete devices. The program code or code segments may represent procedures, functions, subprograms, routines, subroutines, modules, software packages, classes, or any combination of instructions, data structures, or program statements. The program code or code segments may be connected to other code segments or hardware circuitry by sending and receiving information, data, arguments, parameters, or memory contents. The program according to an exemplary embodiment of the present application may also be provided as a program product.

In the exemplary embodiments above, the term “processor” is broad enough to encompass one processor or plural processors in collaboration which are located physically apart from each other but may work cooperatively. The order of operations by the processor is not solely limited to the order described in the exemplary embodiments above, and may be changed, as appropriate.

The “system” in the exemplary embodiments refers to both a configuration formed by multiple devices and a configuration formed by a single device.

The foregoing description of the exemplary embodiments of the present disclosure has been provided for the purposes of illustration and description. It is not intended to be exhaustive or to limit the disclosure to the precise forms disclosed. Obviously, many modifications and variations will be apparent to practitioners skilled in the art. The embodiments were chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

### Appendix

(((1)))

An information processing system comprising a processor configured to:
continuously capture images that include a monitored person;
perform skeletal estimation of the person in a captured image to detect whether or not the person is in an abnormal pose;
if an abnormal pose is detected, input the image in which the abnormal pose of the person is detected into a large language model that converts the content of an inputted image into text information and outputs the text information, and thereby acquire text information pertaining to the state of the person in the inputted image; and
provide a notification indicating that an abnormal pose of the monitored person has occurred, together with the acquired text information and the image in which the abnormal pose is detected, to a preset destination.
   (((2)))

The information processing system according to (((1))), wherein:
the abnormal pose to be detected by skeletal estimation is a fall by the monitored person.
   (((3)))

The information processing system according to (((2))), wherein:
the processor is configured to input the image in which the fall by the monitored person is detected and images before and after the fall into the large language model, and thereby acquire text information containing information pertaining to the state of the monitored person before and after the fall.
   (((4)))

The information processing system according to (((1))), wherein the processor is configured to:
if an abnormal pose of the person is detected by skeletal estimation, determine whether or not the detected abnormal pose is a fall by the monitored person;
if the detected abnormal pose is determined not to be a fall by the monitored person, input the image in which the abnormal pose is detected into the large language model, and thereby acquire text information pertaining to the state of the person in the inputted image; and
provide a notification indicating that an abnormal pose of the monitored person has occurred, together with the acquired text information and the image in which the abnormal pose is detected, to a preset destination.
   (((5)))

The information processing system according to (((4))), wherein the processor is configured to:
if the detected abnormal pose is determined to be a fall by the monitored person, input the image in which the fall by the monitored person is detected and images before and after the fall into the large language model, and thereby acquire text information containing information pertaining to the state of the monitored person before and after the fall; and
provide a notification indicating that a fall by the monitored person has occurred, together with the acquired text information and the image in which the fall is detected, to a preset destination.
   (((6)))

A program causing a computer to execute a process comprising:
continuously capturing images that include a monitored person;
performing skeletal estimation of the person in a captured image to detect whether or not the person is in an abnormal pose;
if an abnormal pose is detected, inputting the image in which the abnormal pose of the person is detected into a large language model that converts the content of an inputted image into text information and outputs the text information, and thereby acquiring text information pertaining to the state of the person in the inputted image; and
providing a notification indicating that an abnormal pose of the monitored person has occurred, together with the acquired text information and the image in which the abnormal pose is detected, to a preset destination.

What is claimed is:

1. An information processing system comprising:
a processor configured to:
continuously capture images that include a monitored person;
perform skeletal estimation of the person in a captured image to detect whether or not the person is in an abnormal pose;
if an abnormal pose is detected, input the image in which the abnormal pose of the person is detected into a large language model that converts the content of an inputted image into text information and outputs the text information, and thereby acquire text information pertaining to the state of the person in the inputted image; and
provide a notification indicating that an abnormal pose of the monitored person has occurred, together with the acquired text information and the image in which the abnormal pose is detected, to a preset destination.

2. The information processing system according to claim 1, wherein:
the abnormal pose to be detected by skeletal estimation is a fall by the monitored person.

3. The information processing system according to claim 2, wherein:
the processor is configured to input the image in which the fall by the monitored person is detected and images before and after the fall into the large language model, and thereby acquire text information containing information pertaining to the state of the monitored person before and after the fall.

4. The information processing system according to claim 1, wherein the processor is configured to:
if an abnormal pose of the person is detected by skeletal estimation, determine whether or not the detected abnormal pose is a fall by the monitored person;
if the detected abnormal pose is determined not to be a fall by the monitored person, input the image in which the abnormal pose is detected into the large language model, and thereby acquire text information pertaining to the state of the person in the inputted image; and
provide a notification indicating that an abnormal pose of the monitored person has occurred, together with the acquired text information and the image in which the abnormal pose is detected, to a preset destination.

5. The information processing system according to claim 4, wherein the processor is configured to:
if the detected abnormal pose is determined to be a fall by the monitored person, input the image in which the fall by the monitored person is detected and images before and after the fall into the large language model, and thereby acquire text information containing information pertaining to the state of the monitored person before and after the fall; and
provide a notification indicating that a fall by the monitored person has occurred, together with the acquired text information and the image in which the fall is detected, to a preset destination.

6. An information processing method comprising:
continuously capturing images that include a monitored person;
performing skeletal estimation of the person in a captured image to detect whether or not the person is in an abnormal pose;
if an abnormal pose is detected, inputting the image in which the abnormal pose of the person is detected into a large language model that converts the content of an inputted image into text information and outputs the text information, and thereby acquiring text information pertaining to the state of the person in the inputted image; and
providing a notification indicating that an abnormal pose of the monitored person has occurred, together with the acquired text information and the image in which the abnormal pose is detected, to a preset destination.

7. A non-transitory computer readable medium storing a program causing a computer to execute a process comprising:
continuously capturing images that include a monitored person;
performing skeletal estimation of the person in a captured image to detect whether or not the person is in an abnormal pose;
if an abnormal pose is detected, inputting the image in which the abnormal pose of the person is detected into a large language model that converts the content of an inputted image into text information and outputs the text information, and thereby acquiring text information pertaining to the state of the person in the inputted image; and
providing a notification indicating that an abnormal pose of the monitored person has occurred, together with the acquired text information and the image in which the abnormal pose is detected, to a preset destination.
